(19) United States
(12) Patent Application Publication
(10) Pub. No.: US 2014/0155633 A1
(43) Pub. Date: Jun. 5, 2014

(54) Composition for Stabilizing Ascorbic Acid Derivatives and the Application Thereof

(71) Applicant: CORUM INC., Taipei City (TW)

(72) Inventors: Lin-Chao Chen, Taipei City (TW); Yu-Ling Shiu, Taipei City (TW); Chia-Pei Lee, Taipei City (TW); Chao-Yang Lee, Taipei City (TW)

(73) Assignee: CORUM INC., Taipei City (TW)

(21) Appl. No.: 13/689,971

(22) Filed: Nov. 30, 2012

(51) Int. Cl. C07D 307/33

(52) U.S. Cl. CPC C07D 307/33
USPC 549/204

(57) ABSTRACT

This invention discloses a composition for stabilizing ascorbic acid derivative and the application thereof. The mentioned composition comprises ascorbic acid derivative, buffer, phosphonic acid derivative and at least one alcohol. The yellowish and degradation of ascorbic acid derivative can be efficiently decreased by the mentioned composition. Moreover, the mentioned composition can be used in topical composition, such as toner, serum, lotion, cream.

### BACKGROUND OF THE INVENTION

1. Field of the Invention

The present invention is generally related to an ascorbic acid derivatives composition, and more particularly to a composition for stabilizing ascorbic acid derivatives and the application thereof.

2. Description of the Prior Art

Ascorbic acid, a.k.a. (also called as) vitamin C, is a water-soluble antioxidant. In addition to its anti-oxidation property, vitamin C is not only recognized for its ability to protect human body from harmful effects of free radicals and environmental pollutants (including CO, hydrocarbons, pesticides and heavy metals), but also for its property to protect DNA of human cells from the damage caused by free radicals and mutagens. Another important function of vitamin C is to strengthen skin tissues through the formation and maintenance of collagens, which helps reduce the expression of wrinkles and delay skin ageing. Especially in the cosmetic industry, vitamin C is also identified to help in the metabolism of tyrosine by inhibiting melanization and preventing skin darkening, thus making it an effective whitening/lightening agent for human skin. Furthermore, vitamin C plays a significant role in many biological functions of human body, as reported in the article “Biological Significance of Ascorbic Acid (Vitamin C) in Human Health—A Review”, published in Pakistan Journal of Nutrition 3(1):5-13, 2004.

Despite all its benefits, vitamin C is extremely unstable; it can be easily oxidized and degraded by oxygen, light, alkali, metals, and high temperature.

In order to stabilize ascorbic acid, a special container for a composition containing ascorbic acid and a hydrophilic carrier which are packaged separately but mixed together upon use was developed in U.S. Pat. No. 6,010,706. This technology of mixing two components, from 0.001 to 0.1 grams of ascorbic acid per gram of carrier, ensures that ascorbic acid does not break and remains stable at room temperature for at least one week. In other words, if this container is not being used, the vitamin C stability will be very poor.

U.S. Pat. No 5,140,043 discloses a composition of ascorbic acid in water and propylene glycol with a pH value of less than 3.5. However, such a low pH could severely irritate human skin, and in some countries, cosmetic laws even prohibit the use of pH lower than 3.5.

U.S. Pat. No. 5,736,567 discloses a composition which contains ascorbic acid dissolved in water and at least one alcohol, forming an aqueous phase, wherein alcohol is present in a quantity that is effective for obtaining a water activity value of ≦0.85. The amount of alcohol(s) used is preferably 45-80% by weight, which is considered a large amount of alcohol(s) in the composition.

U.S. Pat. No. 8,053,469 indicates a production technology that helps stabilize high content of ascorbic acid. This process involves sequential additions of vitamin C, ethoxydiglycol and propylene glycol into the initial solution of vitamin C dissolved in approx. 10% water; and vitamin C at high content has to be divided and added into the solution in several sequences. Nevertheless, the solution also contains a large amount of propylene glycol.

U.S. Pat. No. 6,087,393 discloses a stabilized system of ascorbic acid in a mixed glycol solution. This mixed glycol carrier contains a mixture of propylene glycol and butylene glycol at 25-80% by weight and 5-30% by weight, respectively. Likewise, this composition also contains a high level of propylene glycol.

The four U.S. patents mentioned above can help to improve the stability of vitamin C in various formulations, but there are still some concerns over the use of high concentration of propylene glycol in cosmetic formulations. The North American Contact Dermatitis Group currently recommends a 10% aqueous propylene glycol solution for patch testing, because allergic sensitization has been confirmed by several repeated patch tests, usage tests and oral provocation tests in selected cases. In particular, a significant number of reactions to propylene glycol represent a primary irritant effect. From the studies listed in the article “Propylene glycol dermatitis”, published in Journal of the American Academy of Dermtaology 1991; 24:90-5, it is also clear that there are an increasing amount of irritant reactions when propylene glycol is used in higher concentrations. However, controversies still exist on the potential of allergic sensitizations and irritant reactions caused by this substance.

Furthermore, U.S. Pat. No. 6,110,476 describes a synergistic system based on a phosphonic acid derivative and metabisulfite to stabilize ascorbic acid. However, sodium metabisulfite has been reported as a contact allergen and also as a cause of allergic contact dermatitis in the article “Sodium metabisulfite as a contact allergen—an example of a rare chemical mechanism for protein modification”, published in 2012 John Wiley & Sons A/S•Contact Dermatitis, 66, 123-127. This compound also has a faint SO₂ odor that is unpleasant and pungent to human noses.

3-O-ethyl ascorbic acid is a vitamin C derivative consisting of a conventional vitamin C structure and an additional ethyl group, which makes it more stable than vitamin C. 3-O-ethyl ascorbic acid is tested and recognized for its outstanding ability to inhibit free radical activity, inhibit tyrosinase activity, inhibit melanin production, stimulate collagen synthesis, protected DNA and clinically whiten/lighten/brighten skin tone. Many of these properties have been reported in details by Jill Hsu in the article “New multi-functional and stable vitamin C for skin lightening”, published in NutraCos Cosmetics May/August 2012, p. 6-7.

In addition, another important property of 3-O-ethyl ascorbic acid has been identified in U.S. Pat. No. 2003/0134264A1, which discloses a method of preventing darkening of skin or inhibiting melanization of melanin monomer and a polymerization inhibitor of biological dihydroxyindole compound. The polymerization inhibitor 3-O-ethyl ascorbic acid inhibits the polymerization of a biological dihydroxyindole compound, caused by long wavelength of UVA, and thus reduces melanization significantly.

Although 3-O-ethyl ascorbic acid has a better stability than ascorbic acid, the complete stability of this ascorbic acid derivative hasn't yet been proven and remains unknown up till now.

In view of the above matters, developing a novel ascorbic acid derivative composition having the advantage of stabilizing ascorbic acid derivative and being able to be used in topical composition is still an important task for the industry.

### SUMMARY OF THE INVENTION

In light of the above background, in order to fulfill the requirements of the industry, the present invention provides a novel ascorbic acid derivative composition and the application thereof having the advantage of stabilizing ascorbic acid derivative with mild condition, so that the mentioned composition can be employed in topical composition, such as toner, serum, lotion, cream.

One objective of the present invention is to provide a composition for stabilizing ascorbic acid derivative to reduce the degradation of the ascorbic acid derivative therein.

Another objective of the present invention is to provide a composition for stabilizing ascorbic acid derivative to minimize the color change of the ascorbic acid derivative composition.

Still another objective of the present invention is to provide a composition for stabilizing ascorbic acid derivative. The mentioned composition does not comprise high concentration alcohol therein, so that the composition of this specification can be potentially employed in cosmetics and dermatologic fields without allergic sensitizations and irritant reactions.

Accordingly, the present invention discloses a composition for stabilizing ascorbic acid derivative and the application thereof. The mentioned composition for stabilizing ascorbic acid derivative comprises ascorbic acid derivative, buffer, phosphonic acid derivative, and at least one alcohol. The alcohol must be compatible with water, be polar with one or more hydroxyl groups, and be acceptable for cosmetic use. According to this invention, the mentioned composition can efficiently minimize the color change of the ascorbic acid derivative solution, and efficiently reduce the degradation of the ascorbic acid derivative. We find out that ascorbic acid derivative can be separately stabilized by adjusting the pH value of the composition, adding few amount of phosphonic acid derivative, or adding few amount of at least one alcohol. Preferably, the composition for stabilizing ascorbic acid derivative can be potentially applied in cosmetics and dermatologic fields without allergic sensitizations and irritant reactions to human skin.

### BRIEF DESCRIPTION OF THE DRAWINGS

The present disclosure can be described by the embodiments given below. It is understood, however, that the embodiments below are not necessarily limitations to the present disclosure, but are used to a typical implementation of the invention.

FIG. 1 shows a bar chart of using different alcohol and different amount of alcohol for stabilizing ascorbic acid derivative solution of this invention.

### DESCRIPTION OF THE PREFERRED EMBODIMENTS

What probed into the invention is a composition for stabilizing ascorbic acid derivatives and the application thereof. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment according to this specification discloses a composition for stabilizing ascorbic acid derivative. The mentioned composition comprises ascorbic acid derivative, buffer, phosphonic acid derivative, and at least one alcohol. The general formula of the mentioned ascorbic acid derivative is as the following.

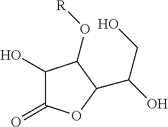

In the above-mentioned formula, R is selected from one of the group consisting of the following: C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group. In one preferred example of this embodiment, the mentioned ascorbic acid derivative is 3-O-ethyl ascorbic acid with the structure as following.

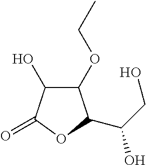

The mentioned buffer is employed to adjust pH of the composition. Preferably, pH of the composition is between 3.5 and 5.5. More preferably, pH of the composition is between 3.8 and 4.5. The mentioned buffer is selected from one of the group consisting of the following: citric acid/sodium citrate (pH 3.0-6.2), citric acid/sodium phosphate (pH 2.6-7.6), sodium acetate/acetic acid (pH 3.7-5.6). In one preferred example of this embodiment, the mentioned buffer is citric acid/sodium citrate (pH 3.0-6.2).

The mentioned phosphonic acid derivative is selected from one of the group consisting of the following: N,N,N′,N′-ethylenediaminetetrakis(methylenephosphonic acid) hydrate (EDTMP), hexaMethylenediaminetetra (methylenephosphonic Acid) (HMDTMPA), Diethylene Triamine Penta (Methylene Phosphonic Acid) (DTPMPA) and the salts thereof.

The mentioned alcohol is selected from one or the combination of the group consisting of the following: ethanol, glycerin, propylene glycol, 1,3-propanediol, dipropylene glycol, butylene glycol, ethoxydiglycol, and polyethylene glycol (PEG). In one preferred example, the average molecular weight of polyethylene glycol is about from 100 to 600 g/mole.

In one preferred example of this embodiment, the mentioned composition for stabilizing ascorbic acid derivative comprises the ascorbic acid derivative from 0.01 to 10% of the total weight of the composition. Preferably, the quantity of the ascorbic acid derivative is from 0.1 to 4.0% of the total weight of the composition. In one preferred example of this embodiment, the mentioned composition for stabilizing ascorbic acid derivative comprises the phosphonic acid derivative from 0.01 to 1.0% of the total weight of the composition. Preferably, the quantity of the phosphonic acid derivative is from 0.1 to 0.5% of the total weight of the composition. In one preferred example of this embodiment, the mentioned composition for stabilizing ascorbic acid derivative comprises the mentioned alcohol not more than 20% of the total weight of the composition. Preferably, the quantity of the alcohol is not more than 10% of the total weight of the composition. In the mentioned composition, the composition further comprises buffer and solvent so that the total weight of the composition approaches 100%. In one preferred example of this embodiment, the solvent is water.

The preferred examples of the structure and fabricating method for the composition for stabilizing ascorbic acid derivatives and the application thereof according to the invention are described in the following. However, the scope of the invention should be based on the claims, but is not restricted by the following examples.

In the following examples, the transmittance is measured by UV-Vis spectrophotometer. The measuring device is Thermo MULTISKAN GO, and the wavelength is set on 440 nm. The general measuring procedure is as the following. A cuvette loaded with distilled water is put into the device for calibration as zero. And then the cuvette loaded with sample is put into the device for measuring the absorbance at 25° C. The transmittance of the sample can be calculated by the following formula.

A=−log T

or written as: T %=10^{-A+2}

Wherein A is absorbance, and T is transmittance (hereinafter presented transmittance as T %). When the measured transmittance of the sample is lower, the sample is more yellow.

The activity of ascorbic acid derivative is also measured by HPLC (High Performance Liquid Chromatography) in this specification. The measuring device is Agilent 1260 HPLC: Quat pump/ALS/TCC/DAD; Column: Prodigy/ODS-3/00F-4097-E0/4.6*150 mm. A bi-solvent system is employed as the mobile phase, the flow rate is set as 1 mL/min, and the detector at 245 nm. In the bi-solvent system, solution A is 0.1% TFA (trifluoroacetic acid)/Acetonitrile, and solution B is 0.1% TFA/double distilled water. Each sample injection is 10 μL. The mobile phase is performed as gradient elution at 25° C., and the gradient program is as the following.

| Time (min) | Solution A (%) | Solution B (%) |
|---|---|---|
| 0.00 | 2 | 98 |
| 10.00 | 98 | 2 |
| 20.00 | 98 | 2 |
| 20.01 | 2 | 98 |
| 35.00 | 2 | 98 |

The total run time is 35 minutes for each injection. And the retention time of the sharp target peak appears on 5.3 minute, while the ascorbic acid derivative is 3-O-ethyl ascorbic acid. The integral of the target peak area is employed for representing the content of ascorbic acid derivative in the sample.

### EXAMPLE 1

For testing the pH decline, 3-O-ethyl ascorbic acid is dissolved in water, and the aqueous solution is placed at 45° C. for 90 days. The test result is presented as the following Table 1. In Entry 1, 1 g 3-O-ethyl ascorbic acid was dissolved in purified water to 100 g form 1% (w/w) solution. In Entry 2, 3 g 3-O-ethyl ascorbic acid was dissolved in purified water to 100 g form 3% (w/w) solution. In Entry 3, 2 g 3-O-ethyl ascorbic acid, 1.49 g sodium citrate and 0.74 g Citric acid were dissolved in purified water to 100 g. In the above experiments, the total amount of the sample that contains the appropriate amount of the preservative.

TABLE 1

| pH | Entry 1 | Entry 2 | Entry 3 |
|---|---|---|---|
| D 0 | 3.44 | 3.47 | 4.73 |
| D 90 | 2.61 | 2.58 | 4.51 |
| D 90 − D 0 | −0.83 | −0.89 | −0.22 |

As shown in Entry 3 in Table 1, buffer system is helpful to stabilize the pH of 3-O-ethyl ascorbic acid solution.

### EXAMPLE 2

In this example, we try to find out the relationship between the pH value and the transmittance (color change) of ascorbic acid derivative solution. In this example, the following solutions were placed at 45° C. for 90 days, and the transmittance of the solutions on Day 0 and Day 90 were respectively detected. Table 2 presents the result of this example. In Entry 4, 0 g 3-O-ethyl ascorbic acid, 1.49 g sodium citrate and 0.74 g Citric acid were dissolved in purified water to 100 g as blank experiment. The pH value of the mentioned blank experiment is 4.73. In Entry 5, 2 g 3-O-ethyl ascorbic acid, 1.49 g sodium citrate and 0.74 g Citric acid were dissolved in purified water to 100 g. The pH value of the solution is 4.73. In Entry 6, 2 g 3-O-ethyl ascorbic acid, 1.44 g sodium citrate and 0.98 g Citric acid were dissolved in purified water to 100 g. The pH value of the solution is 4.42. In the above experiments, the total sample contains the appropriate amount of the preservative. In this example, different pH values (4.73 and 4.46) from the same buffer system were employed. And, the transmittance is detected at 440 nm.

TABLE 2

| Transmittance | | | |
|---|---|---|---|
| T % (at 440 nm) | Entry 4 | Entry 5 | Entry 6 |
| pH value on D 0 | 4.73 | 4.73 | 4.42 |
| D 0 | 99.8 | 99.0 | 99.89 |
| D 90 | 99.8 | 86.6 | 94.69 |
| D 90 − D 0 | −0.0 | −12.4 | −5.2 |

As shown in Entry 5 and Entry 6 in Table 2, it can be found that lower pH value is helpful to stabilize the color of 3-O-ethyl ascorbic acid solution.

### EXAMPLE 3

In this example, we try to compare the stability of ascorbic acid and ascorbic acid derivative solution with buffer. In this example, the following solutions were placed at 45° C. for 9 days, and the transmittance of the solutions on Day 0 and Day 9 were respectively detected. Table 3 presents the result of this example. In Entry 7, 2 g 3-O-ethyl ascorbic acid, 1.28 g sodium citrate, 1.08 g citric acid and appropriate amount of preservative were dissolved in purified water to 100 g. The pH value of the mentioned 3-O-ethyl ascorbic acid solution is 4.22. In Entry 8, 2 g L-ascorbic acid, 1.84 g sodium citrate, 0.70 g Citric acid and appropriate amount of preservative were dissolved in purified water to 100 g. The pH value of the mentioned L-ascorbic acid solution is 4.17. In this example, the transmittance is detected at 440 nm.

TABLE 3

| Transmittance | | |
|---|---|---|
| T % (at 440 nm) | Entry 7 | Entry 8 |
| pH value on D 0 | 4.22 | 4.17 |
| D 0 | 98.81 | 99.00 |
| D 9 | 98.81 | 28.73 |
| D 9 − D 0 | −0.00 | −70.27 |

As shown in Table 3, according to the color change of the samples, it is obviously that 3-O-ethyl ascorbic acid is more stable than L-ascorbic acid.

### EXAMPLE 4

In this example, we try to use phosphonic acid derivative to assist stabilizing ascorbic acid derivative solution. In this example, the following solutions were placed at 45° C. for 90 days, and the transmittance of the solutions on Day 0 and Day 90 were respectively detected at 440 nm. Table 4 presents the result of this example. In Entry 9, 1.44 g sodium citrate, 0.98 g Citric acid and appropriate amount of preservative were dissolved in purified water to 100 g as blank experiment. The pH value of the mentioned blank experiment is 4.42. In Entry 10, 2 g 3-O-ethyl ascorbic acid, 1.44 g sodium citrate, 0.98 g citric acid and appropriate amount of preservative were dissolved in purified water to 100 g. The pH value of the Entry 10 solution is 4.42. In Entry 11, 2 g 3-O-ethyl ascorbic acid, 1.52 g sodium citrate, 0.94 g citric acid, 0.1 g N,N,N,N-tetrakismethylene phosphonate hydrate (EDTMP) and appropriate amount of preservative were dissolved in purified water to 100 g. The pH value of the Entry 11 solution is 4.42.

TABLE 4

| Transmittance | | | |
|---|---|---|---|
| T % (at 440 nm) | Entry 9 | Entry 10 | Entry 11 |
| pH value on D 0 | 4.42 | 4.42 | 4.42 |
| D 0 | 99.8 | 99.89 | 99.11 |
| D 90 | 99.8 | 94.69 | 97.01 |
| D 90 − D 0 | −0.0 | −5.2 | −2.1 |

As shown in Entry 10 and Entry 11 in Table 4, according to the color change of the samples, it can be found that EDTMP is helpful to stabilize 3-O-ethyl ascorbic acid solution.

### EXAMPLE 5

In this example, we try to use different concentration of alcohols to stabilize ascorbic acid derivative solution. In this example, the following solutions were placed at 45° C. for 90 days, and the transmittance of each solution on Day 0 and Day 90 were respectively detected at 440 nm. Table 6 presents the result of this example. In Entry 12, 1.49 g sodium citrate and 0.74 g Citric acid were dissolved in purified water to 100 g as blank experiment. In Entry 13, 2 g 3-O-ethyl ascorbic acid, 1.49 g sodium citrate and 0.74 g citric acid were dissolved in purified water to 100 g. In Entry 14, 2 g 3-O-ethyl ascorbic acid, 1.49 g sodium citrate and 0.74 g citric acid were dissolved in purified water to 100 g, and then 10 g ethoxydiglycol was added into purified water to form a mixed well solution. In Entry 15, 2 g 3-O-ethyl ascorbic acid, 1.62 g sodium citrate and 0.80 g citric acid were dissolved in purified water to 100 g, and then 3.0 g butylene glycol was added into the mentioned purified water to form a mixed well solution. In the above experiments, the total sample contains the appropriate amount of the preservative.

TABLE 5

| Transmittance | | | | |
|---|---|---|---|---|
| T % (at 440 nm) | Entry 12 | Entry 13 | Entry 14 | Entry 15 |
| pH value on D 0 | 4.73 | 4.73 | 4.89 | 4.76 |
| D 0 | 99.8 | 99.0 | 97.6 | 98.8 |
| D 90 | 99.8 | 86.6 | 88.4 | 85.8 |
| D 90 − D 0 | −0.0 | −12.4 | −9.2 | −13.0 |

### EXAMPLE 6

In this example, we try to use phosphonic acid derivative and low concentration alcohols to stabilize ascorbic acid derivative solution. In this example, the following solutions were placed at 45° C. for 90 days, and the transmittance of the solutions on Day 0 and Day 90 were respectively detected at 440 nm. Table 6A presents the result of this example. In Entry 16, 1.44 g sodium citrate and 0.98 g citric acid were dissolved in purified water to 100 g as blank experiment. In Entry 17, 2 g 3-O-ethyl ascorbic acid, 1.44 g sodium citrate and 0.98 g citric acid were dissolved in purified water to 100 g. In Entry 18, 2 g 3-O-ethyl ascorbic acid, 1.52 g sodium citrate, 0.94 g citric acid, and 0.1 g EDTMP were dissolved in purified water to 100 g. In Entry 19, 2 g 3-O-ethyl ascorbic acid, 1.28 g sodium citrate, 0.93 g citric acid, 0.1 g EDTMP and 10.0 g ethoxydiglycol were dissolved in purified water to 100 g. In Entry 20, 2 g 3-O-ethyl ascorbic acid, 1.38 g sodium citrate, 0.95 g citric acid 0.1 g EDTMP and 5.0 g ethoxydiglycol were dissolved in purified water to 100 g. In Entry 21, 2 g 3-O-ethyl ascorbic acid, 1.48 g sodium citrate, 0.90 g citric acid, 0.1 g EDTMP and 3.0 g butylene glycol were dissolved in purified water to 100 g. In the above experiments, the total sample contains the appropriate amount of the preservative.

TABLE 6A

| T % | Entry | Entry | Entry | Entry | Entry | Entry |
|---|---|---|---|---|---|---|
| (at 440 nm) | 16 | 17 | 18 | 19 | 20 | 21 |
| pH value | 4.42 | 4.42 | 4.42 | 4.42 | 4.43 | 4.42 |
| on D 0 | | | | | | |
| T % | 99.8 | 99.89 | 99.11 | 99.47 | 99.86 | 99.45 |
| on D 0 | | | | | | |
| T % | 99.8 | 94.69 | 97.01 | 98.69 | 97.91 | 98.42 |
| On D 90 | | | | | | |
| D 90 − D 0 | −0.0 | −5.2 | −2.1 | −0.80 | −1.95 | −1.03 |

From the above Table 6A, we can find that EDTMP and alcohol are helpful for stabilizing 3-O-ethyl ascorbic acid base on the Transmittance change of the samples.

In order to compare with ascorbic acid, we also process the same test on L-ascorbic acid. The result is shown in the following Table 6B. In Entry 22, 2 g L-ascorbic acid, 1.84 g sodium citrate and 0.70 g citric acid were dissolved in purified water to 100 g. In Entry 23, 2 g L-ascorbic acid, 1.94 g sodium citrate, 0.62 g citric acid, and 0.1 g EDTMP were dissolved in purified water to 100 g. In Entry 24, 2 g L-ascorbic acid, 1.66 g sodium citrate, 0.62 g citric acid, 0.1 g EDTMP and 10.0 g ethoxydiglyco were dissolved in purified water to 100 g. In the above experiments, the total sample contains the appropriate amount of the preservative.

TABLE 6B

| T % | | | |
|---|---|---|---|
| (at 440 nm) | Entry 22 | Entry 23 | Entry 24 |
| pH value | 4.17 | 4.22 | 4.19 |
| on D 0 | | | |
| T % | 99.75 | 99.40 | 98.29 |
| on D 0 | | | |
| T % | 28.73 | 24.59 | 35.83 |
| On D 90 | | | |
| D 90 − D 0 | −71.02 | −74.81 | −62.46 |

From the above Table 6B, as shown in the Transmittance data, we can find that EDTMP and alcohol are not helpful for stabilizing L-ascorbic acid.

### EXAMPLE 7

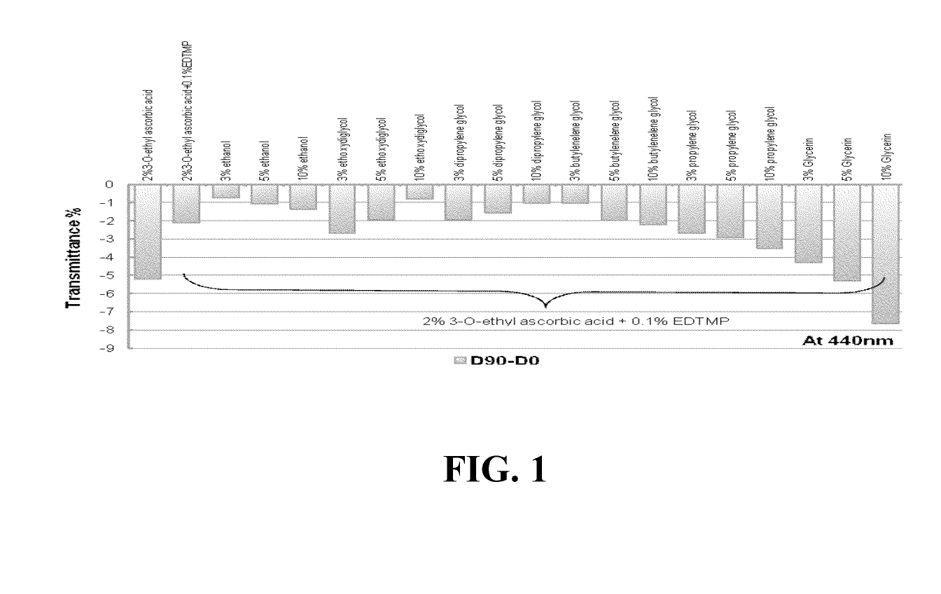

In this example, we try to use different alcohol and different amount of alcohol for stabilizing ascorbic acid derivative solution. In this example, the following solutions were placed at 45° C. for 90 days, and the transmittance of the solutions on Day 0 and Day 90 were respectively detected at 440 nm. The pH values of the solution in this example were controlled at 4.42. Table 7 and FIG. 1 present the result of this example. In Entry 25, 2 g 3-O-ethyl ascorbic acid, 1.44 g sodium citrate, and 0.98 g citric acid were dissolved in purified water to 100 g. In Entry 26, 2 g 3-O-ethyl ascorbic acid, 1.52 g sodium citrate, 0.94 g citric acid, and 0.1 g EDTMP were dissolved in purified water to 100 g. In Entry 27, 2 g 3-O-ethyl ascorbic acid, 1.46 g sodium citrate, 0.95 g citric acid, 0.1 g EDTMP and 3.0 g ethanol were dissolved in purified water to 100 g. In Entry 28, 2 g 3-O-ethyl ascorbic acid, 1.38 g sodium citrate, 0.93 g citric acid, 0.1 g EDTMP and 5.0 g ethanol were dissolved in purified water to 100 g. In Entry 29, 2 g 3-O-ethyl ascorbic acid, 1.30 g sodium citrate, 0.94 g citric acid, 0.1 g EDTMP and 10.0 g ethanol were dissolved in purified water to 100 g. In Entry 30, 2 g 3-O-ethyl ascorbic acid, 1.51 g sodium citrate, 0.95 g citric acid, 0.1 g EDTMP and 3.0 g ethoxydiglycol were dissolved in purified water to 100 g. In Entry 31, 2 g 3-O-ethyl ascorbic acid, 1.38 g sodium citrate, 0.95 g citric acid, 0.1 g EDTMP and 5.0 g ethoxydiglycol were dissolved in purified water to 100 g. In Entry 32, 2 g 3-O-ethyl ascorbic acid, 1.28 g sodium citrate, 0.93 g citric acid, 0.1 g EDTMP and 10.0 g ethoxydiglycol were dissolved in purified water to 100 g. In Entry 33, 2 g 3-O-ethyl ascorbic acid, 1.48 g sodium citrate, 0.90 g citric acid, 0.1 g EDTMP and 3.0 g dipropylene glycol were dissolved in purified water to 100 g. In Entry 34, 2 g 3-O-ethyl ascorbic acid, 1.40 g sodium citrate, 0.93 g citric acid, 0.1 g EDTMP and 5.0 g dipropylene glycol were dissolved in purified water to 100 g. In Entry 35, 2 g 3-O-ethyl ascorbic acid, 1.30 g sodium citrate, 0.88 g citric acid, 0.1 g EDTMP and 10.0 g dipropylene glycol were dissolved in purified water to 100 g. In Entry 36, 2 g 3-O-ethyl ascorbic acid, 1.48 g sodium citrate, 0.90 g citric acid, 0.1 g EDTMP and 3.0 g butylenelene glycol were dissolved in purified water to 100 g. In Entry 37, 2 g 3-O-ethyl ascorbic acid, 1.48 g sodium citrate, 0.93 g citric acid, 0.1 g EDTMP and 5.0 g butylenelene glycol were dissolved in purified water to 100 g. In Entry 38, 2 g 3-O-ethyl ascorbic acid, 1.30 g sodium citrate, 0.88 g citric acid, 0.1 g EDTMP and 10.0 g butylenelene glycol were dissolved in purified water to 100 g. In Entry 39, 2 g 3-O-ethyl ascorbic acid, 1.41 g sodium citrate, 0.95 g citric acid, 0.1 g EDTMP and 3.0 g propylene glycol were dissolved in purified water to 100 g. In Entry 40, 2 g 3-O-ethyl ascorbic acid, 1.38 g sodium citrate, 0.93 g citric acid, 0.1 g EDTMP and 5.0 g propylene glycol were dissolved in purified water to 100 g. In Entry 41, 2 g 3-O-ethyl ascorbic acid, 1.38 g sodium citrate, 0.93 g citric acid, 0.1 g EDTMP and 10.0 g propylene glycol were dissolved in purified water to 100 g. In Entry 42, 2 g 3-O-ethyl ascorbic acid, 1.48 g sodium citrate, 0.90 g citric acid, 0.1 g EDTMP and 3.0 g glycerin were dissolved in purified water to 100 g. In Entry 43, 2 g 3-O-ethyl ascorbic acid, 1.53 g sodium citrate, 0.95 g citric acid, 0.1 g EDTMP and 5.0 g glycerin were dissolved in purified water to 100 g. In Entry 44, 2 g 3-O-ethyl ascorbic acid, 1.40 g sodium citrate, 0.88 g citric acid, 0.1 g EDTMP and 10.0 g glycerin were dissolved in purified water to 100 g. In the above experiments, the total sample contains the appropriate amount of the preservative.

TABLE 7

| T % | | | | | |
|---|---|---|---|---|---|
| (at 440 nm) | Entry 25 | Entry 26 | Entry 27 | Entry 28 | Entry 29 |
| pH value | 4.42 | 4.42 | 4.41 | 4.41 | 4.41 |
| on D 0 | | | | | |
| pH value | 4.20 | 4.22 | 4.21 | 4.20 | 4.22 |
| on D 90 | | | | | |
| pH value | −0.22 | −0.20 | −0.20 | −0.21 | −0.19 |
| D 90 − D 0 | | | | | |
| T % | 99.89 | 99.11 | 98.61 | 98.67 | 98.95 |
| on D 0 | | | | | |
| T % | 94.69 | 97.01 | 97.86 | 97.59 | 97.57 |
| On D 90 | | | | | |
| T % | −5.20 | −2.10 | −0.75 | −1.08 | −1.38 |
| D 90 − D 0 | | | | | |
| HPLC A % | 102.32 | 103.24 | 102.27 | 102.05 | 101.57 |
| on D 0 | | | | | |
| HPLC A % | 86.66 | 90.43 | 96.70 | 87.41 | 91.14 |
| on D 90 | | | | | |
| HPLC A % | −15.66 | −12.81 | −5.57 | −14.64 | −10.43 |
| D 90 − D 0 | | | | | |
| T % | | | | | |
| (at 440 nm) | Entry 30 | Entry 31 | Entry 32 | Entry 33 | Entry 34 |
| pH value | 4.43 | 4.43 | 4.42 | 4.43 | 4.40 |
| on D 0 | | | | | |
| pH value | 4.19 | 4.22 | 4.24 | 4.22 | 4.18 |
| on D 90 | | | | | |
| pH value | −0.24 | −0.21 | −0.18 | −0.21 | −0.22 |
| D 90 − D 0 | | | | | |
| T % | 99.99 | 99.86 | 99.47 | 99.78 | 99.00 |
| on D 0 | | | | | |
| T % | 97.32 | 97.91 | 98.67 | 97.85 | 97.43 |
| On D 90 | | | | | |
| T % | −2.67 | −1.95 | −0.80 | −1.93 | −1.57 |
| D 90 − D 0 | | | | | |
| HPLC A % | 102.61 | 102.75 | 102.55 | 103.68 | 101.55 |
| on D 0 | | | | | |
| HPLC A % | 94.82 | 97.11 | 92.98 | 90.84 | 93.00 |
| on D 90 | | | | | |
| HPLC A % | −7.79 | −5.64 | −9.57 | −12.84 | −8.55 |
| D 90 − D 0 | | | | | |
| T % | | | | | |
| (at 440 nm) | Entry 35 | Entry 36 | Entry 37 | Entry 38 | Entry 39 |
| pH value | 4.41 | 4.42 | 4.42 | 4.41 | 4.40 |
| on D 0 | | | | | |
| pH value | 4.21 | 4.24 | 4.25 | 4.26 | 4.16 |
| on D 90 | | | | | |
| pH value | −0.20 | −0.18 | −0.17 | −0.15 | −0.24 |
| D 90 − D 0 | | | | | |
| T % | 99.22 | 99.45 | 99.13 | 99.33 | 99.45 |
| on D 0 | | | | | |
| T % | 98.19 | 98.42 | 97.16 | 97.11 | 96.87 |
| On D 90 | | | | | |
| T % | −1.03 | −1.03 | −1.97 | −2.22 | −2.67 |
| D 90 − D 0 | | | | | |
| HPLC A % | 101.57 | 103.76 | 102.31 | 101.30 | 100.99 |
| on D 0 | | | | | |
| HPLC A % | 91.02 | 91.42 | 88.50 | 88.79 | 87.84 |
| on D 90 | | | | | |
| HPLC A % | −10.55 | −12.34 | −13.81 | −12.51 | −13.15 |
| D 90 − D 0 | | | | | |
| T % | | | | | |
| (at 440 nm) | Entry 40 | Entry 41 | Entry 42 | Entry 43 | Entry 44 |
| pH value | 4.40 | 4.41 | 4.42 | 4.43 | 4.42 |
| on D 0 | | | | | |
| pH value | 4.17 | 4.25 | 4.21 | 4.21 | 4.19 |
| on D 90 | | | | | |
| pH value | −0.23 | −0.16 | −0.21 | −0.22 | −0.23 |
| D 90 − D 0 | | | | | |
| T % | 99.45 | 99.03 | 99.06 | 98.76 | 98.45 |
| on D 0 | | | | | |
| T % | 96.52 | 95.49 | 94.78 | 93.45 | 90.80 |
| On D 90 | | | | | |
| T % | −2.93 | −3.54 | −4.28 | −5.31 | −7.64 |
| D 90 − D 0 | | | | | |
| HPLC A % | 104.14 | 102.82 | 102.68 | 103.00 | 102.55 |
| on D 0 | | | | | |
| HPLC A % | 88.88 | 91.76 | 86.18 | 85.64 | 85.08 |
| on D 90 | | | | | |
| HPLC A % | −15.26 | −11.06 | −16.50 | −17.36 | −17.47 |
| D 90 − D 0 | | | | | |

As shown in Table 7, buffer system is helpful to stabilize the pH of 3-O-ethyl ascorbic acid solution. As shown in Entry 27 in Table 7, adding 0.1% EDTMP and 3% ethanol is helpful to stabilize color of 3-O-ethyl ascorbic acid solution. As shown in Entry 28 in Table 7, adding 0.1% EDTMP and 5% ethanol is helpful to stabilize color of 3-O-ethyl ascorbic acid solution. As shown in Entry 29 in Table 7, adding 0.1% EDTMP and 10% ethanol is helpful to stabilize color of 3-O-ethyl ascorbic acid solution. As shown in Entry 32 in Table 7, adding 0.1% EDTMP and 10% ethoxydiglycol is helpful to stabilize color of 3-O-ethyl ascorbic acid solution. As shown in Entry 35 in Table 7, adding 0.1% EDTMP and 10% dipropylene glycol is helpful to stabilize color of 3-O-ethyl ascorbic acid solution. As shown in Entry 36 in Table 7, adding 0.1% EDTMP and 3% butylenelene glycol is helpful to stabilize color of 3-O-ethyl ascorbic acid solution. As shown in Entry 27 to 38 in Table 7, adding 0.1% EDTMP and ethanol/ethoxydiglycol/dipropylene glycol/butylenelene glycol is helpful to delay degradation of 3-O-ethyl ascorbic acid solution.

### EXAMPLE 8

In this example, we try to use two alcohol in different ratios to stabilize ascorbic acid derivative solution. In this example, the following solutions were placed at 45° C. for 90 days, and the transmittance of the solutions on Day 0 and Day 90 were respectively detected at 440 nm. The pH values of the solution in this example were controlled at 4.2. In this example, in order to check the stability of ascorbic acid derivative, we used transmittance of ascorbic acid derivative solution to follow the yellowing. Furthermore, we also used HPLC to check the activity of ascorbic acid derivative from the change of the area integral of the 3-O-ethyl ascorbic acid peak in HPLC assay. Table 8 presents the result of this example. In Entry 45, 2 g 3-O-ethyl ascorbic acid, 1.35 g sodium citrate, 1.04 g citric acid, and 0.1 g EDTMP were dissolved in purified water to 100 g. In Entry 46, 2 g 3-O-ethyl ascorbic acid, 1.29 g sodium citrate, 1.05 g citric acid, 0.1 g EDTMP and 2.0 g ethanol were dissolved in purified water to 100 g. In Entry 47, 2 g 3-O-ethyl ascorbic acid, 1.25 g sodium citrate, 1.04 g citric acid, 0.1 g EDTMP 4.0 g and ethanol were dissolved in purified water to 100 g. In Entry 48, 2 g 3-O-ethyl ascorbic acid, 1.29 g sodium citrate, 1.05 g citric acid, 0.1 g EDTMP and 2.0 g butylene glycol were dissolved in purified water to 100 g. In Entry 49, 2 g 3-O-ethyl ascorbic acid, 1.25 g sodium citrate, 1.04 g citric acid, 0.1 g EDTMP and 4.0 g butylene glycol were dissolved in purified water to 100 g. In Entry 50, 2 g 3-O-ethyl ascorbic acid, 1.21 g sodium citrate, 1.02 g citric acid, 0.1 g EDTMP 3.0 g and ethanol and 3.0 g butylene glycol were dissolved in purified water to 100 g. In the above experiments, the total sample contains the appropriate amount of the preservative.

TABLE 8

| T % | Entry | Entry | Entry | Entry | Entry | Entry |
|---|---|---|---|---|---|---|
| (at 440 nm) | 45 | 46 | 47 | 48 | 49 | 50 |
| pH value | 4.23 | 4.20 | 4.21 | 4.19 | 4.20 | 4.21 |
| on D 0 | | | | | | |
| pH value | 4.08 | 4.06 | 4.10 | 4.03 | 4.08 | 4.11 |
| On D 90 | | | | | | |
| pH value | −0.15 | −0.14 | −0.11 | −0.16 | −0.12 | −0.10 |
| D 90 − D 0 | | | | | | |
| T % | 99.33 | 99.36 | 99.56 | 99.40 | 99.01 | 98.56 |
| on D 0 | | | | | | |
| T % | 97.56 | 97.88 | 98.42 | 98.11 | 98.40 | 98.40 |
| On D 90 | | | | | | |
| T % | −1.74 | −1.48 | −1.14 | −1.29 | −0.61 | −0.16 |
| D 90 − D 0 | | | | | | |
| HPLC A % | 100.11 | 100.00 | 100.42 | 100.02 | 100.30 | 100.03 |
| on D 0 | | | | | | |
| HPLC A % | 85.98 | 87.80 | 86.97 | 82.03 | 88.66 | 88.92 |
| on D 90 | | | | | | |
| HPLC A % | −14.13 | −12.20 | −13.45 | −7.99 | −11.64 | −11.11 |
| D 90 − D 0 | | | | | | |

From the above Table 8, we can find that adding 0.1% EDTMP, 3% Ethanol and 3% 1,3-Butylene Glycol are helpful for stabilizing 3-O-ethyl ascorbic acid, based on the measured Transmittance data and HPLC assay. And, it also can be found that phosphonic acid derivative and the at least one alcohol can be synergistic on stabilizing 3-O-ethyl ascorbic acid.

### EXAMPLE 9

### Application of the Composition of Stabilizing 3-O-Ethyl Ascorbic Acid in Toner

The following is the major components of three entries with the composition of stabilizing 3-O-ethyl ascorbic acid according to this specification.

| | Ingredient | Entry 51 |
|---|---|---|
| A | Purified water | To 100.00 |
| | Allantoin | 0.10 |
| | Sodium Citrate | 0.95 |
| | N,N,N,N-Ethylenediaminetetrakis | 0.10 |
| | (Methylenephosphonic Acid) | |
| | Citric Acid | 1.05 |
| | Ethoxydiglycol | 10.00 |
| | Ethanol | 3.00 |
| | Jojoba Wax PEG-120 Esters | 0.50 |
| | Sodium Hyaluronate (1%) | 1.00 |
| | Preservative | q.s. |
| | 3-O-ethyl ascorbic acid | 1.00 |

| | |
|---|---|
| Appearance | clear |
| pH value(25° C.) | 4.11 |

In this example, the manufacturing of the above-mentioned toners is as the following. The part A are mixed homogeneously.

### EXAMPLE 10

### Application of the Composition of Stabilizing 3-O-Ethyl Ascorbic Acid in Serum

The following is the major components of three entries with the composition of stabilizing 3-O-ethyl ascorbic acid according to this specification.

| | Ingredient | Entry 52 |
|---|---|---|
| A | Purified water | To 100.00 |
| | Hydroxyethylcellulose (2%) | 20.00 |
| | Sodium Citrate | 1.00 |
| | N,N,N,N-Ethylenediaminetetrakis | 0.10 |
| | (Methylenephosphonic Acid) | |
| | Citric acid | 0.99 |
| | Ethoxydiglycol | 10.00 |
| | 1,3-Butylene Glycol | 3.00 |
| | Jojoba Wax PEG-120 Esters | 0.50 |
| | Sodium Hyaluronate (1%) | 1.00 |
| | Preservative | q.s. |
| | 3-O-ethyl ascorbic acid | 2.00 |

| | |
|---|---|
| Appearance | clear |
| pH value (25° C.) | 4.31 |
| Viscosity | 157.2 cPs |
| (S63/1000 rpm/25° C./60 sec) | |

In this example, the manufacturing of the above-mentioned serums is as the following. The part A are mixed homogeneously.

### EXAMPLE 11

### Application of the Composition of Stabilizing 3-O-Ethyl Ascorbic Acid in Lotion

The following is the major components of the entry with the composition of stabilizing 3-O-ethyl ascorbic acid according to this specification.

| | INCI Name | Entry 53 |
|---|---|---|
| A | Emulsifier | 6.00 |
| A | Cetearyl Alcohol | 1.0 |
| A | Lauryl/Myristyl Benzoate | 3.00 |
| A | Octyldodecyl Myristate | 5.00 |
| A | Dioctyl Sebacate | 5.00 |
| A | Macadamia Integrifolia Nut Oil | 1.00 |
| A | Dimethicone | 1.00 |
| B | Purified water | To 100.00 |
| B | 1,3-Butylene Glycol | 3.00 |
| B | Citric acid | 0.71 |
| B | Sodium Citrate | 1.17 |
| B | N,N,N,N-Ethylenediaminetetrakis | 0.20 |
| | (Methylenephosphonic Acid) | |
| C | Preservative | q.s. |
| C | Fragrance | 0.15 |
| D | Purified water | 5.00 |
| D | 3-O-ethyl ascorbic acid | 2.00 |

| | |
|---|---|
| Appearance | White |
| pH value (25° C.) | 4.29 |
| Viscosity (S64/30 rpm/25° C./30 sec) | 7,100 |

In this example, the manufacturing of the above-mentioned lotion is as the following. The part A, part B, part C and part D are pre-mixed separately. Part A and part B are heated up to 80° C. Then, part B is added into part A, and the mixture of part A and part B are mixed well homogeneously. The mentioned mixture of part A and part B are stirred for 5 minutes, and then the mixture is removed from the heat source. When the mixture is cooled down to 40° C., part C and part D are added into the mixture sequentially to form a well mixed.

### EXAMPLE 12

### Application of the Composition of Stabilizing 3-O-Ethyl Ascorbic Acid in Cream

The following is the major components of the entry with the composition of stabilizing 3-O-ethyl ascorbic acid according to this specification.

| | INCI Name | Entry 54 |
|---|---|---|
| A | Emulsifier | 6.00 |
| A | Lauryl/Myristyl Benzoate | 3.00 |
| A | 2-Octyldodecyl Myristate | 5.00 |
| A | Octyldodecyl Stearoyl Stearate | 5.00 |
| A | Cetearyl Alcohol | 2.00 |
| A | Macadamia Integrifolia Nut Oil | 1.00 |
| A | Dimethicone | 1.00 |
| B | Purified water | To 100.00 |
| B | Microcrystalline Cellulose (and) Cellulose Gum | 1.50 |
| C | Sodium Citrate | 0.60 |
| C | N,N,N′,N′-Ethylenediaminetetrakis | 0.10 |
| | (methylenephosphonic Acid) Hydrate | |
| C | Citric acid | 0.82 |
| C | 1,3-Butylene Glycol | 3.00 |
| D | Potassium Azelaoyl Diglycinate | 5.00 |
| D | Preservative | q.s. |
| D | Fragrance | 0.05 |
| E | Purified water | 3.00 |
| E | 3-O-ethyl ascorbic acid | 3.00 |
| E | Alcohol | 3.00 |

| | |
|---|---|
| Appearance | White |
| pH Value (25° C.) | 4.25 |
| Viscosity (S64/12 rpm/25° C./30 Sec) | 22,500 cPs |

In this example, the manufacturing of the above-mentioned cream is as the following. Part A, part B, part C, part D, and part E are pre-mixed separately. Part C is added into part B, and the mixture is well-mixed. Part A and the mixture of part B/C are respectively heated up to 80° C. Then, the mixture of part B/C is added into part A and mixed well. The mentioned mixture of part A/B/C are stirred for 5 minutes, and then the mixture is removed from the heat source. When cooling the mixture of part A/B/C down to 40° C., part D and part E are added into the mentioned mixture sequentially, and mixed well.

### EXAMPLE 13

### Application of the Composition of Stabilizing 3-O-Ethyl Ascorbic Acid in Cream

The following is the major components of the entry with the composition of stabilizing 3-O-ethyl ascorbic acid according to this specification.

| | INCI Name | Entry 55 |
|---|---|---|
| A | Emulsifier | 6.00 |
| A | Lauryl/Myristyl Benzoate | 3.00 |
| A | 2-Octyldodecyl Myristate | 5.00 |
| A | Octyldodecyl Stearoyl Stearate | 5.00 |
| A | Cetearyl Alcohol | 2.00 |
| A | Macadamia Integrifolia Nut Oil | 1.00 |
| A | Dimethicone | 1.00 |
| B | Purified water | To 100.00 |
| B | Microcrystalline Cellulose (and) Cellulose Gum | 1.50 |
| C | Sodium Citrate | 0.58 |
| C | N,N,N′,N′-Ethylenediaminetetrakis | 0.10 |
| | (methylenephosphonic Acid) Hydrate | |
| C | Citric acid | 0.82 |
| C | 1,3-Butylene Glycol | 3.00 |
| D | Potassium Azelaoyl Diglycinate | 5.00 |
| D | Preservative | q.s. |
| D | Fragrance | 0.05 |
| E | Alcohol | 3.00 |
| E | Purified water | 3.00 |
| E | 3-O-ethyl ascorbic acid | 4.00 |

| | |
|---|---|
| Appearance | White |
| pH Value(25° C.) | 4.28 |
| Viscosity (S64/30 rpm/25° C./30 Sec) | 13,617 cPs |

The manufacturing of the above-mentioned cream is as the following. Part B is pre-mixed and heated up to 75° C. The ingrediants in part C are added into part B in sequence, and the mixture of part B and part C is mixed well. Part A is pre-mixed, and heated up to 75° C. The mixture of part B/C is added into part A. The mentioned mixture of part A/B/C is stirred for 5 minutes, and then the mixture is removed from the heat source. When cooling the mixture of part A/B/C down to 40° C., the ingredients of part D and pre-mixed part E are added into the mentioned mixture sequentially, and mixed well.

### EXAMPLE 14

### Application of the Composition of Stabilizing 3-O-Ethyl Ascorbic Acid in Cream

The following is the major components of the entry with the composition of stabilizing 3-O-ethyl ascorbic acid according to this specification.

| | INCI Name | Entry 56 |
|---|---|---|
| A | Emulsifier | 7.00 |
| A | Lauryl/Myristyl Benzoate | 3.00 |
| A | 2-Octyldodecyl Myristate | 5.00 |
| A | Octyldodecyl Stearoyl Stearate | 5.00 |
| A | Cetearyl Alcohol | 1.50 |
| A | Macadamia Integrifolia Nut Oil | 1.00 |
| A | Dimethicone | 1.00 |
| B | Purified water | To 100.00 |
| B | Xanthan Gum | 0.35 |
| C | Sodium Citrate | 0.51 |
| C | N,N,N′,N′-Ethylenediaminetetrakis | 0.10 |
| | (methylenephosphonic Acid) Hydrate | |
| C | Citric acid | 0.74 |
| C | 1,3-Butylene Glycol | 3.00 |
| C | Ethoxydiglycol | 10.00 |
| D | Preservative | q.s. |
| D | Fragrance | 0.05 |
| E | Purified water | 1.50 |
| E | Ethanol | 3.00 |
| E | 3-O-ethyl ascorbic acid | 7.00 |

| | |
|---|---|
| Appearance | White |
| pH Value(25° C.) | 4.05 |
| Viscosity (S64/3 rpm/25° C./30 Sec) | 95,380 cPs |

The manufacturing of the above-mentioned cream is as the following. Part B is pre-mixed and heated up to 75° C. The ingredients in part C are added into part B in sequence, and the mixture of part B/C is mixed well. Part A is pre-mixed, and heated up to 75° C. The mixture of part B/C is added into part A. The mentioned mixture of part A/B/C is stirred for 5 minutes, and then the mixture is removed from the heat source. When cooling the mixture of part A/B/C down to 40° C., the ingredients of part D and pre-mixed part E are added into the mentioned mixture sequentially, and mixed well.

### EXAMPLE 15

### Application of the Composition of Stabilizing 3-O-Ethyl Ascorbic Acid in Cream (W/O)

The following is the major components of the entry with the composition of stabilizing 3-O-ethyl ascorbic acid according to this specification.

| | INCI Name | Entry 57 |
|---|---|---|
| A | Polyglyceryl-3 Diisostearate | 1.67 |
| A | Polyglyceryl-6 Dioleate | 3.33 |
| A | Isohexadecane | 4.00 |
| A | Isododecane | 3.00 |
| A | Dimethicone | 3.00 |
| A | Petrolatum | 4.00 |
| A | Paraffinium Liquidum (and) Disteardimonium | 6.00 |
| | (and) Hectorite (and) Propylene Carbonate | |
| A | Preservative | q.s. |
| A | Fragrance | 0.15 |
| B | Water | Add to 100.00 |
| B | Sodium Citrate | 0.76 |
| B | N,N,N′,N′-Ethylenediaminetetrakis(methylene- | 0.10 |
| | phosphonic Acid) Hydrate | |
| B | Citric acid | 0.69 |
| B | 3-O-ethyl ascorbic acid | 2.00 |
| B | 1,3-Butylene Glycol | 3.00 |

| | |
|---|---|
| Appearance | Slightly |
| | yellowish |
| | cream |
| pH Value of water base (25° C.) | 4.20 |
| Viscosity (S64/30 rpm/25° C./30 Sec) | 9,998 cPs |

The manufacturing of the above-mentioned cream is as the following. Part A is pre-mixed and well-blended. Part B is pre-mixed. Part B is slowly (drop by drop) added into part A while well-stirring.

### EXAMPLE 16

### Application of the Composition of Stabilizing 3-O-Ethyl Ascorbic Acid in Cream (W/O/W)

The following is the major components of the entry with the composition of stabilizing 3-O-ethyl ascorbic acid according to this specification.

Primary Emulsion:

| | INCI Name | Entry 58 |
|---|---|---|
| A | PEG-30 Dipolyhydroxystearate | 2.15 |
| A | Jojoba Oil | 8.00 |
| A | Helianthus Annuus (Sunflower) Seed Oil | 7.00 |
| B | Water | 29.21 |
| B | N,N,N′,N′-Ethylenediaminetetrakis | 0.05 |
| | (methylene-phosphonic Acid)Hydrate | |
| B | Citric acid | 0.37 |
| B | Sodium Citrate | 0.37 |
| B | 3-O-ethyl ascorbic acid | 2.00 |
| B | 1,3-Butylene Glycol | 3.00 |
| C | Fragrance | 0.05 |
| C | Preservative | 0.80 |

| | |
|---|---|
| Appearance | White |
| pH Value of inner aqueous phase (25° C.) | 4.09 |
| Viscosity (S63/30 rpm/25° C./30 Sec) | 2,200 cPs |

Secondary Emulsion:

| | | |
|---|---|---|
| D | Water | 43.60 |
| D | Polysorbate-80 | 2.00 |
| D | Sclerotium Gum | 1.00 |
| D | Xanthan Gum | 0.30 |
| D | Methylisothiazolinone | 0.10 |

| | |
|---|---|
| Appearance | White |
| pH Value of outer aqueous phase (25° C.) | 6.14 |
| Viscosity (S63/30 rpm/25° C./30 Sec) | 91,980 cPs |

The manufacturing of the above-mentioned cream is as the following. Firstly, in the Primary Emulsion, part B is pre-mixed and heated up to 30° C. Part A is heated up to 60° C. Part B is slowly (drop by drop) added into part A while well-stirring (stirring rate is 700 rpm) for 10 minutes. Then, part C is added into the mixture of part A/B for 10 minutes. The mixture of part A/B/C is homogenized at 8000 rpm for 10 minutes to produce the Primary Emulsion. Subsequently, in the Secondary Emulsion, part D is pre-mixed. Pre-mixed part D is slowly added into part A/B/C while well-stirring (stirring rate is 1000 rpm) for 10 minutes. The mixture of part A/B/C/D is stirred for 5 minutes at 800 rpm, and then stirred for 5 minutes at 500 rpm.

In summary, we have reported a composition for stabilizing ascorbic acid derivative and the application thereof. The composition comprises ascorbic acid derivative, buffer, phosphonic acid derivative and at least one alcohol. According to this invention, we find out that the stability of ascorbic acid derivative can be improved by adding buffer, phosphonic acid derivative, or alcohol separately. We also find out that when forming a composition comprising buffer, phosphonic acid derivative, and alcohol, the stabilizing effect can be synergistic. Preferably, all the components in the mentioned composition for stabilizing ascorbic acid derivative are not expensive, so that it will not raise the cost too much while employing the mentioned composition to replace L-ascorbic acid in cosmetics and dermatologic fields. More preferably, the composition is mild, so that it can be applied in cosmetics and dermatologic fields without allergic sensitizations and irritant reactions to human skin.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A clear composition for stabilizing ascorbic acid derivative comprising:
an ascorbic acid derivative, wherein a general formula of the ascorbic acid derivative is as the following, 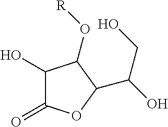
wherein R is selected from one of the group consisting of the following: C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group;
a buffer, wherein the buffer is employed to adjust pH value of the composition between 3.5 and 5.5; and
a stabilizing agent consisted of phosphonic acid derivative and alcohol, wherein said alcohol is selected from one or the combination of the group consisting of the following: ethanol, propylene glycol, 1,3-propanediol, dipropylene glycol, butylene glycol, ethoxydiglycol, and polypolyethylene glycol (PEG).

2. The clear composition for stabilizing ascorbic acid derivative according to claim 1, wherein said buffer is selected from one of the group consisting of the following: citric acid/sodium citrate, citric acid/sodium phosphate, and acetic acid/sodium acetate.

3. The clear composition for stabilizing ascorbic acid derivative according to claim 1, wherein said phosphonic acid derivative is selected from one of the group consisting of the following: N,N,N′,N′-ethylenediaminetetrakis(methylenephosphonic acid) hydrate (EDTMP), hexaMethylenediaminetetra (methylenephosphonic Acid) (HMDTMPA), Diethylene Triamine Penta (Methylene Phosphonic Acid) (DTPMPA) and the salts thereof.

4. (canceled)

5. The clear composition for stabilizing ascorbic acid derivative according to claim 1, wherein the quantity of the ascorbic acid derivative is from 0.01 to 10% of the total weight of the composition.

6. The clear composition for stabilizing ascorbic acid derivative according to claim 1, wherein the quantity of the ascorbic acid derivative is from 0.1 to 4.0% of the total weight of the composition.

7. The clear composition for stabilizing ascorbic acid derivative according to claim 1, wherein the quantity of the phosphonic acid derivative is from 0.01 to 1.0% of the total weight of the composition.

8. The clear composition for stabilizing ascorbic acid derivative according to claim 1, wherein the quantity of the phosphonic acid derivative is from 0.1 to 0.5% of the total weight of the composition.

9. The clear composition for stabilizing ascorbic acid derivative according to claim 1, wherein the quantity of the alcohol is not more than 20% of the total weight of the composition.

10. The clear composition for stabilizing ascorbic acid derivative according to claim 1, wherein the quantity of the alcohol is not more than 10% of the total weight of the composition.

11. The clear composition for stabilizing ascorbic acid derivative according to claim 1, wherein the pH value of the composition is between 3.8 and 4.5.

12. The clear composition for stabilizing ascorbic acid derivative according to claim 1, further comprising water.

13. The clear composition for stabilizing ascorbic acid derivative according to claim 1, wherein said ascorbic acid derivative is 3-O-ethyl ascorbic acid with the structure as following.

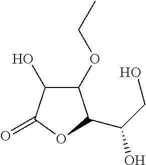

14. A clear composition for stabilizing ascorbic acid derivative comprising:
an ascorbic acid derivative, wherein a general formula of the ascorbic acid derivative is as the following, 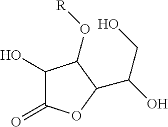
wherein R is selected from one of the group consisting of the following: C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group;
a buffer, wherein the buffer is employed to adjust pH value of the composition between 3.5 and 5.5; and
a stabilizing agent consisted of phosphonic acid derivative and alcohol.
